(19) United States
(12) Patent Application Publication
(10) Pub. No.: US 2022/0331142 A1
(43) Pub. Date: Oct. 20, 2022

(54) IMPOTENCE TREATMENT

(71) Applicant: Adrian PAZ, Petah Tikva (IL)

(72) Inventor: Adrian PAZ, Petah Tikva (IL)

(21) Appl. No.: 17/639,912

(22) PCT Filed: Sep. 9, 2020
(86) PCT No.: PCT/IL2020/050985

§ 371 Date: Mar. 3, 2022

Related U.S. Application Data
(60) Provisional application No. 62/954,746, filed on Dec. 30, 2019.
(60) Provisional application No. 62/898,574, filed on Sep. 11, 2019.

(51) Int. Cl. A61F 5/41; A61N 7/00

(52) U.S. Cl. CPC A61F 5/41; A61N 7/00; A61F 2005/417; A61N 2007/0004

(57) ABSTRACT

The current disclosure relates to a system for treating impotence. In some embodiments, the device includes a mount for holding a transducer in intimate contact to a penile tissue. For example, an ultrasound transducer and/or a vibration transducer may be held in vibrational communication with a corpora, a crura, a root of the penis and/or a glans. For example, the mount may include a sheath and/or a tubular body that may be rigid, partially flexible and/or flexible. The sheath may be open on one or both ends and/or may fully surround the penis and/or include a slit on one side. Optionally, the tubular body includes one or more extensions that reach the root of the penis and/or the crura. For example, the extensions may extend proximally from a proximal end of the sheath and/or the extensions may include transducers that face and/or inward (towards one another).

### RELATED APPLICATION/S

This application claims the benefit of priority under 35 USC § 119(e) of U.S. Provisional Patent Application No. 62/898,574 filed 11 Sep. 2019, the contents of which are incorporated herein by reference in their entirety.

This application claims the benefit of priority under 35 USC § 119(e) of U.S. Provisional Patent Application No. 62/954,746 filed 30 Dec. 2019, the contents of which are incorporated herein by reference in their entirety.

### FIELD AND BACKGROUND OF THE INVENTION

The present invention, in some embodiments thereof, relates to a system for treating impotence and, more particularly, but not exclusively, the system may include an Ultra Sound transducer and/or vibration transducer and/or a portable holder.

U.S. Pat. No. 9,913,748 appears to disclose, “A method and device producing extracorporeal shockwaves for the treatment of erectile dysfunction.”

U.S. patent Ser. No. 10/441,498 appears to disclose that, “Devices and methods for generating acoustic shock wave within a cavity is disclosed. The shock wave device optionally includes a housing having a cylindrical portion and a cone frustum portion. The housing optionally forms a cavity configured to receive a penis. The shock wave device optionally includes a plurality of shock wave generators and a coupling assembly having a deformable sac configured to hold shock wave transmitting liquid. The volume of the transmitting liquid is optionally increased or decreased as needed so that the coupling assembly can conform to the shape of the penis. The shock waves generated optionally has an intensity gradient within the cavity of the shock wave device, where the intensity gradient is optionally controllable using a control and power supply unit.”

Additional background art includes US Patent Application Publication no. 20180296383, International Patent Application Publication no. WO2002030343, U.S. Pat. No. 8,360,956, US Patent Application Publication no. 20140171734, U.S. Pat. No. 5,370,601, US Patent Application Publication no. AU2017232036, and

Xin Z, Lin G, Lei H, Lue T F, Guo Y. Clinical applications of low-intensity pulsed ultrasound and its potential role in urology. Transl Androl Urol. 2016; 5(2):255-266. doi:10.21037/tau. 2016.02.04.

### SUMMARY OF THE INVENTION

According to an aspect of some embodiments of the invention, there is provided a system for treating impotence including: a sheath having a lumen configured to fit around a penis; a transducer directed towards the lumen; at least one more transducer directed towards a penile crura located proximal to the lumen.

According to some embodiments of the invention, the at least one more transducer is directed at least partially in a dorsal direction with respect to the sheath.

According to some embodiments of the invention, the at least one more transducer includes a pair of inward directed transducers.

According to an aspect of some embodiments of the invention, there is provided a system for treating impotence including: a sheath having a lumen configured to fit around a penis; a transducer directed towards the lumen; a proximal extension from the sheath; at least one more transducer held by the extension.

According to some embodiments of the invention, the sheath is elastic for accommodating different penile diameters.

According to some embodiments of the invention, the at extension is configured to hold the at least one more transducer against a crura of the penis when at least a portion of a body the penis is inside the lumen.

According to some embodiments of the invention, the system further includes a vibrator.

According to some embodiments of the invention, the vibrator is positioned on a distalmost ½ of the sheath.

According to some embodiments of the invention, the vibrator is positioned on a dorsal side, proximal to a glans, or on the glans.

According to some embodiments of the invention, the transducer includes a low intensity ultrasound transducer.

According to some embodiments of the invention, the at least one more transducer includes a low intensity ultrasound transducer.

According to some embodiments of the invention, the system further includes a sensor of at least one of rigidity and/or tumescence.

According to some embodiments of the invention, the sensor includes a strain gauge.

According to some embodiments of the invention, the transducer the at least one more transducer are connected to a controller.

According to some embodiments of the invention, the controller is configured to discern a quality of contact between at least one more transducer and tissue.

According to some embodiments of the invention, the controller is configured to discern a quality of contact between the transducer and tissue.

According to some embodiments of the invention, the quality of contact is determined by receiving an ultrasound reflection from the transducer fitted to the lumen and/or the at least one more transducer on the extension.

According to some embodiments of the invention, the system further includes one or more thermal sensors in thermal contact with the transducer fitted to the lumen and/or the at least one more transducer on the extension.

According to some embodiments of the invention, the controller is configured prevent the treating when the quality of contact is poor.

According to some embodiments of the invention, at least one of the transducer the at least one more transducer is configured for delivering between 20 to 500 W/cm2.

According to some embodiments of the invention, the at least one more transducer is configured for delivering a signal of frequency between 1 to 3 MHz.

According to some embodiments of the invention, the vibrator is configured for delivering a signal of frequency between 10 to 300 Hz.

According to some embodiments of the invention, the system further includes at least one fluid filled cushion located between the transducer and the lumen.

According to some embodiments of the invention, the controller includes a hand held electronic device chosen form a group consisting of a cellular phone, a smart watch, a laptop, an iPad and a PDA.

According to some embodiments of the invention, a connection between the transducer and the controller is wireless.

According to some embodiments of the invention, the hand held electronic device includes a program module for receiving data, a program module for analyzing data, a user interface module for displaying data and changing parameters.

According to some embodiments of the invention, the data from different users is anonymized and evaluated by cloud computing in order to improve treatment parameters for individualized treatment.

According to some embodiments of the invention, the sheath further includes a longitudinal slit.

According to some embodiments of the invention, the sheath is provided with straps to tightly compress the sheath over the penis.

According to some embodiments of the invention, one or more cushions are provided on inner surface of a wall of the lumen, to facilitate transmission of ultrasound waves from the transducer to a penile cavernous corpora.

According to some embodiments of the invention, the lumen is provided with an adhesive surface to prevent slippage of the penis.

According to an aspect of some embodiments of the invention, there is provided a method for treating erectile disfunction including: applying over a penis of an appliance including at least one LIPUS transducer positioned in intimate contact with penile tissue of a body of the penis; applying at least one more LIPUS transducer positioned in intimate contact with a crura of the penis; and activating the LIPUS transducer and the at least one more LIPUS transducer for treating or improving erectile dysfunction.

According to some embodiments of the invention, the at least one LIPUS transducer and the at least one more LIPUS transducer are applied simultaneously.

According to some embodiments of the invention, the method further includes: creating penis engorgement resulting in at least a partial erection.

According to some embodiments of the invention, the method further includes: retaining the transducer in contact with the the penile tissue with a sheath enveloping at least part of the body of the penis.

According to some embodiments of the invention, the method further includes: retaining the at least one more transducer in contact with the crura with a proximal extension from the sheath.

According to some embodiments of the invention, the method further includes: discern a quality of contact between a transducer and tissue.

According to some embodiments of the invention, the method further includes: prevent the activating when the quality of contact is poor.

According to some embodiments of the invention, activating includes delivering between 20 to 500 W/cm2.

According to some embodiments of the invention, activating includes delivering a signal of frequency between 1 to 3 MHz.

According to some embodiments of the invention, activating includes delivering signals at a burst cycle frequency of 500 Hz to 2 KHz.

According to some embodiments of the invention, activating includes delivering a vibratory signal of frequency between 10 to 300 Hz.

According to some embodiments of the invention, the system further includes: positioning at least one fluid filled cushion between the transducer and a lumen of the sheath.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of a system and/or an apparatus for treatment of impotence in accordance with an embodiment of the current invention;

FIG. 2 is a flow chart illustration of a method of improving erectile function in accordance with an embodiment of the current invention;

FIG. 3 is a flow chart illustration of a method of positioning a system to improve potency in accordance with an embodiment of the current invention;

FIG. 4A is a schematic dorsal view of a device for impotency treatment in accordance with an embodiment of the current invention;

FIG. 4B is a schematic ventral view of a device for impotency treatment in accordance with an embodiment of the current invention;

FIG. 4C is a schematic distal view of a device for impotency treatment in accordance with an embodiment of the current invention;

FIG. 5 is a schematic distal view of a sheath in accordance with an embodiment of the current invention;

FIG. 6 is a cross section view of a sheath in accordance with an embodiment of the current invention;

FIG. 7A is a schematic distal view of a device for impotency treatment in accordance with an embodiment of the current invention;

FIG. 7B is a schematic lateral view of a device for impotency treatment placed on an erect penis in accordance with an embodiment of the current invention;

FIG. 7C is a schematic lateral view of a device for impotency treatment placed on a flaccid penis in accordance with an embodiment of the current invention;

FIG. 7D is a schematic proximal view of a device for impotency treatment placed on a penis in accordance with an embodiment of the current invention;

FIG. 8 is a schematic illustration of a user preparing to use an impotency treatment device in accordance with an embodiment of the current invention;

FIG. 9 is a schematic illustration of a device for treating impotence in accordance with an embodiment of the current invention;

FIG. 10 is a schematic illustration of a device for treating impotence in accordance with an embodiment of the current invention;

FIG. 11 is a schematic illustration of a device for treating impotence in accordance with an embodiment of the current invention;

FIG. 12A is a schematic illustration of a system for treatment of impotence including an inflatable cushion in an uninflated state in accordance with an embodiment of the current invention;

FIG. 12B is a schematic illustration of a system for treatment of impotence including an inflatable cushion in an inflated state in accordance with an embodiment of the current invention;

FIG. 13 is a schematic illustration of a system for treating impotence in use in accordance with an embodiment of the current invention;

FIG. 14 is a schematic view of an appliance for attaching a transducer to penile tissue in accordance with an embodiment of the current invention;

FIG. 15 illustrates a transducer configured for reversable attachment to a glans in accordance with an embodiment of the current invention;

FIG. 16 illustrates a transducer and/or mount configured for reversible attachment to the penile corpora in accordance with an embodiment of the current invention;

FIG. 17 illustrates a controller for an erectile disfunction treatment system in accordance with an embodiment of the current invention;

FIG. 18A is a schematic illustration of a dorsal side of a device for treatment of impotence including a sheath having a dorsal slit and/or an inflatable cushion in an empty state in accordance with an embodiment of the current invention;

FIG. 18B is a schematic illustration of a dorsal side of a device for treatment of impotence including a sheath having a dorsal slit and/or an inflatable cushion in an inflated state in accordance with an embodiment of the current invention; and

FIG. 18C is a schematic lateral view illustration of a device for treatment of impotence including a sheath having a dorsal slit and/or an inflatable cushion in an inflated state in accordance with an embodiment of the current invention.

### DESCRIPTION OF SPECIFIC EMBODIMENTS OF THE INVENTION

The present invention, in some embodiments thereof, relates to a system for treating impotence and, more particularly, but not exclusively, the system may include an Ultra Sound transducer and/or vibration transducer and/or a portable holder.

### Overview

An aspect of some embodiments of the current invention relates to a system for treating impotence. In some embodiments, the device includes a mount for holding a transducer in intimate contact to a penile tissue. For example, an ultrasound transducer and/or a vibration transducer may be held in vibrational communication with a corpora of the penis and/or the crura and/or a root of the penis and/or a glans. For example, the mount may include a sheath and/or a tubular body that may be rigid, partially flexible and/or flexible. The sheath may be open on one or both ends and/or may fully surround the penis and/or include an longitudinal slit. Optionally, the tubular body includes one or more extensions that reach the root of the penis and/or the crura. For example, the extensions may extend proximally from a proximal end of the sheath and/or the extensions may include transducers that face cranially and/or inward (towards one another). In some embodiments, the extensions may be elastic and/or biased to push a transducer into intimate contact with a crura and/or a penis root. Alternately or additionally, a user may hold the extensions and/or a transducer in intimate contact with a crura and/or a penis root (e.g. by closing his legs to squeeze the extension and/or transducer between his leg and the crura and/or root). The extensions and/or transducers may be configured to contact the crura and/or root when the penis is erect and/or flaccid. Optionally, the sheath includes a support to hold the sheath erect even when the penis is flaccid. For example, the transducers contacting the crura may be directed at an angle between 0 to 5 degrees and/or between 5 to 20 degrees and/or between 20 to 45 degrees and/or between 20 to 45 degrees and/or between 45 to 90 degrees of cranial. Optionally, the extensions may be at an angle of between 0 to 5 degrees and/or between 5 to 20 degrees and/or between 20 to 45 degrees and/or between 20 to 45 degrees and/or between 45 to 90 degrees of axis of the sheath. For example, an extension may extend between 0.5 to 1 cm and/or between 1 to 2 cm and/or between 2 to 5 cm proximal and/or posterior to a proximal end of a sheath.

In some embodiments, the length of a lumen of a sheath may range between 2 to 5 cm and/or between 5 to 7 cm and/or between 7 to 10 cm and/or between 10 to 15 cm and/or between 15 to 20 cm. Optionally there are different sized sheaths for different sized penises.

In some embodiments, the width of a lumen of a sheath when closed may range between 1 to 2 cm and/or between 2 to 4 cm and/or between 4 to 6. In some embodiments, the width of a lumen of a sheath when open may range between 2 to 4 cm and/or between 4 to 6 cm and/or between 6 to 8. Optionally there are different sized sheaths for different sized penises.

In some embodiments, the width of a slit in a lumen of a sheath when closed may range between 0 to 1 cm and/or between 1 to 3 cm and/or between 3 to 6. In some embodiments, the width of a lumen of a sheath when open may range between 1 to 3 cm and/or between 3 to 6 cm and/or between 6 to 8. Optionally there are different sized sheaths for different sized penises.

In some embodiments, the system comprises, a device for creating at least partial erection, comprising a tubular body, closed at one end and open at the other end, that can be fitted over the flaccid penis. The tubular body may be connected to a vacuum device. Optionally, the device is fitted over the flaccid penis and its opening is snugly fit over the perineum and partial vacuum is created. Optionally, the reduced pressure in the tubular body will cause penile engorgement and tumescence creating at least partial erection. In some embodiments, the partial erection may be maintained during treatment by applying a temporary elastic band at the base of the penis. Alternatively or additionally, one or more vibrators may be used to arouse a full or partial erection. For example, the vibrator may be positioned on or near the glans. For example, the vibrator may be positioned on a distal part of a sheath around the penis. For example, the vibrator may be positioned on the distalmost ⅛ and/or distalmost ¼ and/or distalmost ½ of the sheath. In some embodiments, a vibrator may be positioned on the dorsal side of the penis shaft proximal to the glans.

In some embodiments, the system comprises, an appliance such as a condom or a band or tubular body, or an appliance with a central lumen, comprising one or more Low Intensity Pulsed UltraSound (LIPUS) transducers that are in intimate contact with the penile corpora cavernosa and/or the penis crura or roots during treatment. At the end of the treatment, the appliance is optionally removed and/or the elastic band at the base of the penis is removed resulting in detumescence and flaccidity of the penis.

Some embodiments of the current invention relate to a method for treating erectile dysfunction. The method optionally comprises: applying over the penis of an appliance comprising at least one LIPUS transducer positioned in intimate contact with the penile tissue; activating the LIPUS transducer for treating or improving erectile dysfunction. Optionally, penis engorgement in created resulting in at least a partial erection.

In some embodiments, applying a treatment may include applying a signal (e.g. an ultrasound signal) to the penile tissue (e.g. the corpora, the crura, the penile root and/or the glans). For example, the signal strength may range between 10 to 50 mW/cm² and/or between 50 to 100 mW/cm² and/or between 100 to 500 mW/cm² and/or between 500 to 2000 mW/cm². For example, the area of contact of each transducer may range between 0 to 2 cm² and/or between 2 to 6 cm² and/or between 6 to 15 cm². For example, the total signal power may range between 0.4 to 0.8 W and/or between 0.8 to 1.6 W and/or between 1.6 to 4 W and/or between 4 to 20 W. For example a duty cycle of the signal may range between 1 to 5% and/or between 5 to 15% and/or between 15 to 30% and/or between 30 to 60% and/or between 60 to 100%. For example, frequency of cycles may range between 10 Hz to 1 kHz and/or between 1 kHz to 100 kHz. The transducer frequency may range between 500 kHz to 1M, and/or between 1 MHz TO 3 MHz. A treatment session may last for example, between 10 sec to 1 min and/or between 1 to 5 min and/or between 5 to 20 min and/or between 20 min to 2 hours and/or between 2 to 24 hours. Optionally, multiple transducers may operate at a phase difference (some on at one time and/or others on at another time). Optionally, the power supply may include a battery and/or a transformer and/or a rectifier and/or a switched mode power supply. In some embodiments, a battery made supply power for between 1 to 5 sessions and/or between 5 to 20 sessions and/or between 20 to 100 sessions. In some embodiments, a power supply and/or transducer may be configured according to any of the above treatment modularities.

Penile tissue may include for example, the corpora of the penis, the crura and/or roots of the penis. In some embodiments, creating penis engorgement or partial erection may improve the efficacy of the treatment by for example but not limited to: increasing the treated volume, by stretching the cavernous tissue and/or increasing its susceptibility to the non-thermal effects of the ultrasonic waves and/or by acting on the trapped blood cells in the cavernous tissue to release neo-angiogenetic and/or trophic factors.

Creating penis engorgement may be achieved, for example, by any, some or all of the methods: of using a drug such as a PDE5 inhibitor such: SINDENAFIL, VARDENAFIL, TADALFIL, AVANFIL, or using vasoactive agents injected into the cavernous body or applied to mucosa of the urethra, or by using a vacuum device for creating a penile erection and applying a temporary elastic band at the base of the penis, or by using self-stimulation. An elastic band may be applied at the base of the penis, after realizing penile tumescence with any of the mentioned method to prevent detumescence during the treatment. In some embodiments, method may also comprise: actively causing penis detumescence or flaccidity. In case of using of an elastic band this is done by removing the elastic band at the base of the penis.

### Specific Embodiments

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

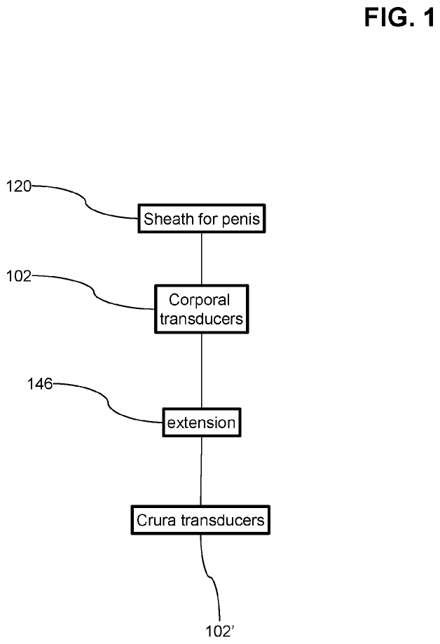

FIG. 1 is a block diagram of a system and/or an apparatus for treatment of impotence in accordance with an embodiment of the current invention. In some embodiments, the device includes a sheath 120 configured to envelope a body of the penis. Optionally, the sheath 120 may hold one or more transducers 102 in intimate contact with a corpora of the penis. In some embodiment, there are one or more extensions 146 extending proximally from the sheath. For example, the extension 146 may be positioned on a ventral portion of the sheath 120. Optionally, the extensions 146 have cranial directed transducers. For example, the extensions 146 may be configured to bring the transducers 102′ into intimate contact with a crura and/or root of the penis.

Additionally or alternatively, the sheath may include a vibrator and/or hold the vibrator to penile tissue. For example, the vibrator may be held to a glans and/or to tissue slightly proximal to the glans.

In some embodiments, the system may include one or more sensors to detect a state of the penis (e.g. rigid, flaccid) and/or a state of the transducers (e.g. in contact with tissue, hot, not in contact, cool).

In some embodiments, the system includes a controller. For example, the controller may control signal (e.g. Ultrasound signals) sent by the transducers. Additionally or alternatively, the processor may receive input from the transducers and/or sensors. For example, the processor may adjust signal properties based on received sensor and/or transducer output.

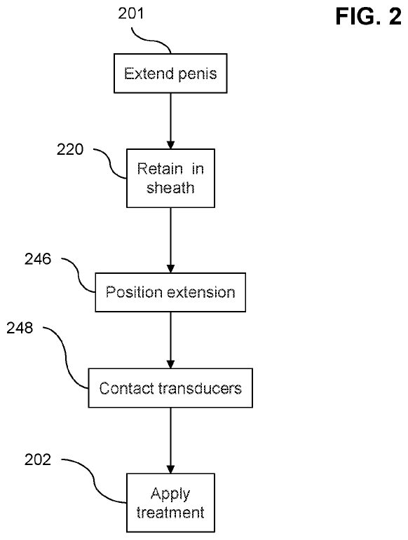

FIG. 2 is a flow chart illustration of a method of improving erectile function in accordance with an embodiment of the current invention. In some embodiments, a method of treating impotence comprises: reversibly attaching of one or more transducers (for example including low intensity ultrasound modules) to the penile corpora and/or to the penile crura). For example, the attachment may be using a distend able cuff or using an adhesive patch.

In some embodiments, a user may extend 201 his penis (for example by pulling and/or longitudinally stretching a flaccid penis and/or by inducing an erection). Optionally, the body of the penis is retained 220 within a sheath. Optionally, the sheath may encompass a portion of the body of the penis with the glans extending outward distally. Alternatively or additionally, a distal portion of the penis may be entirely enveloped by the sheath. Optionally, the sheath may envelope the body of the penis on three sides (e.g. with a slit one or more sides, for example, the dorsal side, the ventral a lateral side. Optionally, the sheath holds transducers (for example ultrasound transducers) in contact 248 with penile tissue (for example the corpora). For example, the transducers may be integrated into the sheath. Alternatively or additionally, the transducers may be placed on the penis and/or the sheath wrapped around them. Contacting 248 the transducers with penile tissue may include for example, tightening the sheath around the penis, providing a contact intermediary (for example Ultrasound gel and/or an ultrasound transmitting pad) and/or engorging the penis to contact the sheath and/or expanding an inner surface of the sheath (e.g. an inflatable cushion) towards the penis. For example, contacting 248 between the transducer and the tissue may operationally be defined as supplying a path for transmission of vibration and/or sound waves between the transducer and the tissue.

In some embodiments, an extension is positioned 246 next to the penis root and/or crura. For example, the extension may include a proximal extension of the of the sheath. Alternatively or additionally, the extension may include a piece of underclothing. Optionally, the extension is positioned 246 to contact 248 one or more transducers (e.g. Ultrasound transducers) with the penile root and/or crura. Optionally, the transducer is held in contact 428 with the root or crura by a clamp and/or by elastic forces due to the shape and position of the extensions. Additionally or alternatively, the transducer may be held in contact with the crura using a piece of underclothing and/or by the user closing his legs to sandwich the transducer between his thighs and the root of his penis.

In some embodiments, a vibrator may be brought in contact with penile tissue. For example, the vibrator may be attached to the glans and/or just behind the glans using a patch or a distend able cuff. Alternatively or additionally, a strain gauge may be positioned and/or attached to penile tissue, for example, behind the glans. Optionally the vibrator may be activated in order to induce some tumescence and rigidity. The level of tumescence and rigidity may be monitored using the strain gauge which may act as a feed-back sensor. In case of attaining a predetermined degree of tumescence and rigidity the vibratory module may be turned off. The transducers are optionally activated to apply 202 a treatment to improve potency in long term or even cure impotency. For example, it the treatment may include predominantly non-thermal effects that effect angiogenesis, vascularization. For example, applying 202 a treatment may include applying 202 a signal (e.g. an ultrasound signal) to the penile tissue (e.g. the corpora, the crura, the penile root and/or the glans). For example, the signal strength may range between 10 to 50 mW/cm² and/or between 50 to 100 mW/cm² and/or between 100 to 500 mW/cm² and/or between 500 to 2000 mW/cm². For example, the area of contact of each transducer may range between 0 to 2 cm² and/or between 2 to 6 cm² and/or between 6 to 15 cm². For example, the total signal power may range between 0.4 to 0.8 W and/or between 0.8 to 1.6 W and/or between 1.6 to 4 W and/or between 4 to 20 W. For example, a duty cycle of the signal may range between 1 to 5% and/or between 5 to 15% and/or between 15 to 30% and/or between 30 to 60% and/or between 60 to 100%. For example, frequency of cycles may range between 10 Hz to 1 kHz and/or between 1 kHz to 100 kHz and/or between 100 kHz to 1 MHz and/or between 1 MHz to 100 MHz. A treatment session may last for example, between 10 sec to 1 min and/or between 1 to 5 min and/or between 5 to 20 min and/or between 20 min to 2 hours and/or between 2 to 24 hours. Optionally, multiple transducers may operate at a phase difference (some on at one time and/or others on at another time). Optionally, the power supply may include a battery and/or a transformer and/or a rectifier and/or a switched mode power supply. In some embodiments, a battery made supply power for between 1 to 5 sessions and/or between 5 to 20 sessions and/or between 20 to 100 sessions.

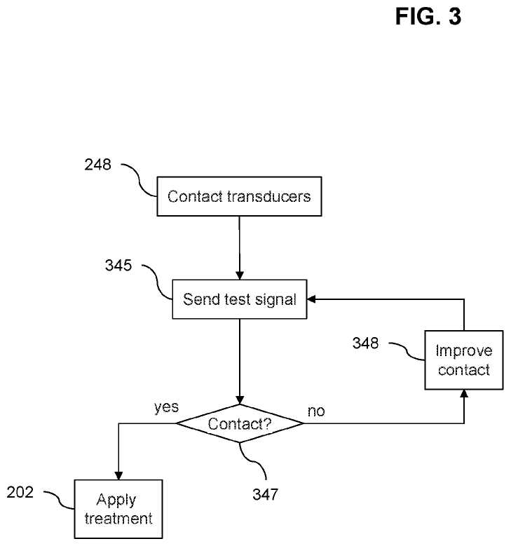

FIG. 3 is a flow chart illustration of a method of positioning a system to improve potency in accordance with an embodiment of the current invention. Optionally, a transducer is positioned and/or brought in contact 248 with penile tissue (for example as described above and/or using any of the steps disclosed in the description of FIG. 2). Optionally, once the transducer is positioned, a test signal is sent 345 from the transducer to the tissue. Data is optionally collected (for example, including output of a sensor (e.g. a thermal sensor), and/or feedback from the transducer (e.g. an ultrasound reflection from the transducer and/or from another transducer). If the contact between the transducer determined 347 to be is good, then the system goes on to apply 202 a treatment. If the contact is determined to be poor (e.g. if there is poor gain and/or unexpected resistance to a signal and/or the transducer is heating up) then the user is warned. Optionally the user improves 348 contact between the transducer and the tissue. For example, the user may reposition the transducer and/or tighten a mount and/or add gel between the transducer and tissue and/or adds a pad between the transducer and tissue. The system may automatically test contact between transducers and tissue and/or may have a safety mechanism that prevents activation of a treatment routine when contact between the transducer and the tissue is poor and/or when a transducer is overheating. For example, in home treatment this may inhibit users burning themselves by improper use of the equipment.

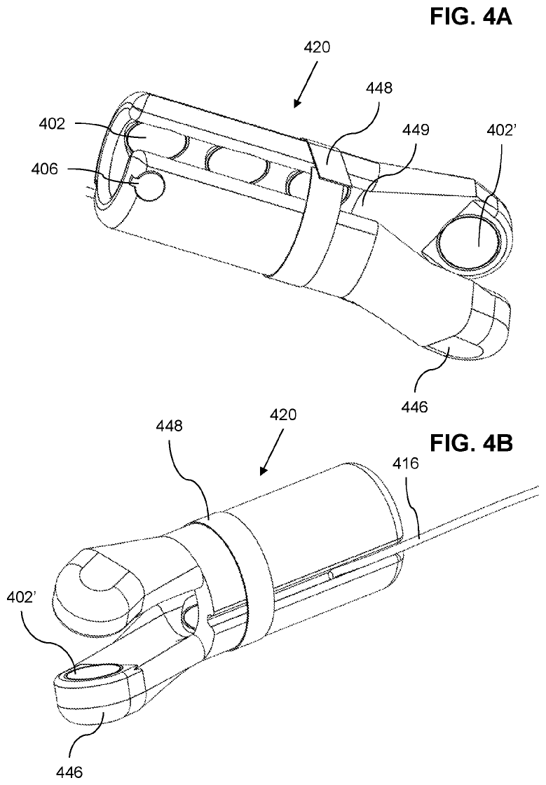

FIG. 4A is a schematic dorsal view of a device for impotency treatment in accordance with an embodiment of the current invention. In some embodiments, a device may include a flexible sheath 420. For example, the sheath may comprise silicone, rubber and/or an elastomer. Optionally the sheath 420 includes a lumen and mounts for retaining transducers 402 (for example six LIPUS transducers 402 and/or between 1 to 3 transducers and/or between 4 to 8 transducers and/or between 8 to 20 transducers) in contact with penile tissue inside the lumen. Optionally there are one or more rows of transducers along each cavernous body. In some embodiments, the shape of the transducers maybe round or oval or rectangular. The size of the transducers may range between 0.1 to 0.5 cm and/or between 0.5 to 1 cm in diameter and/or between 1 to 3 cm in diameter. Optionally, a dorsal slit 449. runs the length of the sheath 420. Alternatively or additionally, a slit may be on any side of the sheath and/or may run along a part of the length thereof. For example, the slit 449 may facilitate inserting a penis into the sheath 420. For example, a flaccid penis may be stretched and/or laid into the sheath.

In some embodiments, the sheath 420 includes a pair of proximal extensions 446. For example, extensions 446 may be configured to retain a pair of transducers 402′ against a root and/or crura. Optionally, there may be more than one transducer on each extension. For example, the number of transducers contacting the root and/or crura may range between one to two, two to four, four to eight and/or eight to twenty. For example, when the sheath is placed on a flaccid and/or erect penis, transducers 402′ may be directed cranially and/or inward. For example, the transducers contacting the crura may be directed at an angle between 0 to 5 degrees and/or between 5 to 20 degrees and/or between 20 to 45 degrees and/or between 20 to 45 degrees and/or between 45 to 90 degrees of cranial. Optionally, the extensions may be at an angle of between 0 to 5 degrees and/or between 5 to 20 degrees and/or between 20 to 45 degrees and/or between 20 to 45 degrees and/or between 45 to 90 degrees of a longitudinal axis of the sheath and/or the lumen of the sheath.

In some embodiments, the device includes one or more straps 448. For example, strap 448 may be used to tighten sheath 420 onto a penis and/or to bring transducers 402 into intimate contact with penile tissue and/or to narrow a longitudinal slit on the sheath. Optionally, sheath 420 includes a vibrator 406 near a distal opening thereof.

FIG. 4B is a schematic ventral view of a device for impotency treatment in accordance with an embodiment of the current invention. In some embodiments, a device may include a cable 416. For example, cable 416 may attach the device to a power source, for example for powering transducers 402, 402′. Alternatively or additionally, the device may include a battery. Optionally, the battery may be rechargeable and/or cable 416 may act as a recharge cable. Optionally cable 426 is permanently connected to the device. Alternatively or additionally, the cable may be reversibly attached to the device. Alternatively or additionally, the device may include a wireless charging module. In some embodiments, the system may include a controller controlling transducers 402, 402′ and/or a receiving data from transducers 402, 402′ and/or sensors. Alternatively or additionally, the device may communicate wirelessly with a controller. Alternatively or additionally, the device may include an internal processor. Optionally the internal processor may communicate wirelessly and/or over a wired connection with an external controller, user interface and/or power source. Alternatively or additionally, the device may include a built in user interface. Alternatively or additionally, all the control module and battery may be incorporated in the mount device. Optionally, a controller is configured to activate different transducers at different times and/or according to different powers and/or work cycles. For example, a transducer on a penile crura and/or root may be activated at different times and/or at the same time as transducers on a corpora.

FIG. 4C is a schematic distal view of a device for impotency treatment in accordance with an embodiment of the current invention. In some embodiments, a device may include a cavity 447 for positioning Ultrasound transmitting gel between a transducer 402 and tissue (for example between the transducer 402 and/or the lumen of the sheath 420.

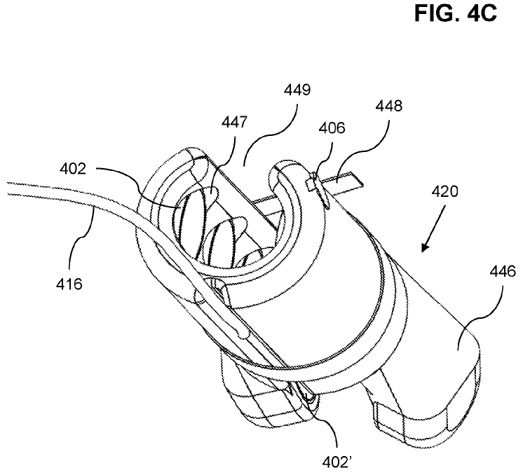

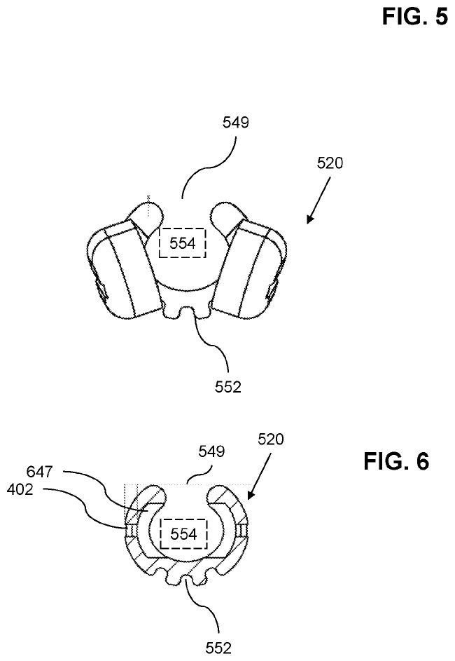

FIG. 5 is a schematic distal view of a sheath in an open configuration in accordance with an embodiment of the current invention. FIG. 6 is a cross section view of a sheath in a closed configuration in accordance with an embodiment of the current invention. Optionally, a sheath 520 may have an open and/or a closed configuration. For example, a slit 549 in the sheath may open to a lumen 554 of the sheath 520. In closed configuration, the slit is optionally smaller than in the open configuration and/or completely closed. Optionally, in an open configuration, the lumen 554 may expand for example to between 1.1 to 1.5 times is closed size and/or 1.5 to 2 times its closed size and/or between 2 to 4 times its closed size. Optionally, in an open configuration, the slit 549 may expand for example to between 1.1 to 1.5 times is closed size and/or 1.5 to 2 times its closed size and/or between 2 to 4 times its closed size. Optionally when the sheath 520 opens and/or closes, the sheath 520 may flex. For example, on the side opposite the slit 549 there may be stress releasing features 552, for example a thinning of wall and/or a groove. In some embodiments, a pad 647 may intervein between a transducer 402 and a lumen 554 and/or between a transducer 402 and a penis. Optionally, the pad 647 may be customizable (e.g. by cutting and/or by replacing with different sized pads and/or by cutting to size). Optionally the pad is made of an Ultrasound transmitting material. The pad is optionally filled with a fluid and/or made of a soft material and/or rigid.

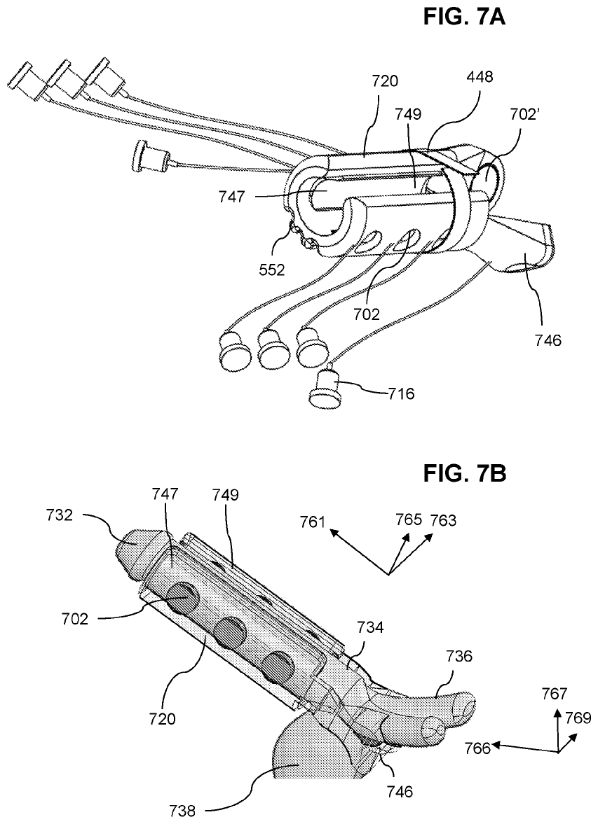

FIG. 7A is a schematic distal view of a device for impotency treatment in accordance with an embodiment of the current invention. In some embodiments, a set of transducers 702 are held facing a lumen of a sheath 720 and/or the penis body 734. Optionally, the transducers are connected to a set of connectors 716, for example connecting to a power supply and/or a controller. Alternatively or additionally, there may be an internal power supply and/or internal controller. Alternatively or additionally, transducers are connected wirelessly to a controller and/or charger. In some embodiment, the lumen includes a dorsal slit 749. Optionally, proximal extensions 746 hold transducers 702′ against a penile root and/or crura.

In some embodiments, a pad 747 may intervein between transducers 702 and the lumen and/or between transducers 702 and a penis. Optionally, the pad 747 may be customizable (e.g. by cutting and/or by replacing with different sized pads and/or by cutting to size). Optionally the pad is made of an Ultrasound transmitting material. The pad is optionally filled with a fluid and/or made of a soft material and/or rigid.

FIG. 7B is a schematic lateral view of a device for impotency treatment placed on an erect penis in accordance with an embodiment of the current invention. Two sets of axes are shown. A sheath centered and/or penis centered set of axesaxes includes a distal proximal axis having a distal direction 761 shown, a dorsal ventral axis having a dorsal direction 765 shown and/or a lateral axis 763. A body centered set of axes includes a cranial caudal axis having a cranial direction 767 shown, an anterior posterior axis having an anterior direction 766 shown and/or a lateral axis 769. Optionally, the proximal extensions are angled according to the position of the penis with respect to the body. For example, when used with the penis is erect, the extensions 746 are angled upward (cranially/dorsally) with respect to the axis of the penis/lumen of the sheath 720. For example, the proximal transducers 702′ face cranially and/or inwardly towards the penile crura 736. The extensions 746 are optionally located on a ventral portion of the sheath 720 and/or spread laterally in the proximal direction facilitating their passing around and/or above a scrotum 738, and/or the extensions are located on the lateral sides of the sheath. Optionally a glans 732 of the penis projects distally out of a distal opening of the sheath 720. Alternatively or additionally, the glans 732 may be contained within the lumen of the sheath 420. In some embodiments, the sheath 720 may be supported by a support structure to remain erect even when the penis is flaccid.

FIG. 7C is a schematic lateral view of a device for impotency treatment placed on a penis in accordance with an embodiment of the current invention. Optionally, for use with a flaccid penis, extensions 746 extend proximally parallel to and/or at a small angle to the axis of the sheath 720. In some embodiments, an angle of the extensions 746 is adjustable. For example, there may be a joint and/or the extensions 746 may be malleable facilitating bending them to keep a transducer 702′ in contact with the penile crura 736 and/or root as the angle of the penis with respect to the body changes. Alternately or additionally, the extensions may be elastic facilitating bending them to keep a transducer 702′ in contact with the penile crura 736 and/or root as the angle of the penis with respect to the body changes.

FIG. 7D is a schematic proximal view of a device for impotency treatment placed on a penis in accordance with an embodiment of the current invention. In the proximal view, the optional cranial inward direction of the proximal transducer 702′ is observable.

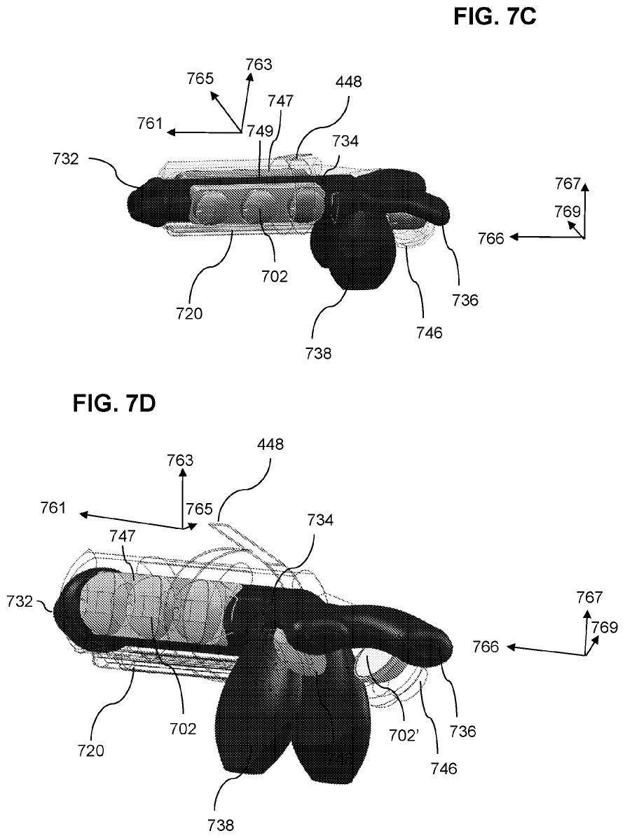

FIG. 8 is a schematic illustration of a user preparing to use an impotency treatment device in accordance with an embodiment of the current invention. In some embodiments, a user may start with flaccid penile tissue (for example penile corpora 834).

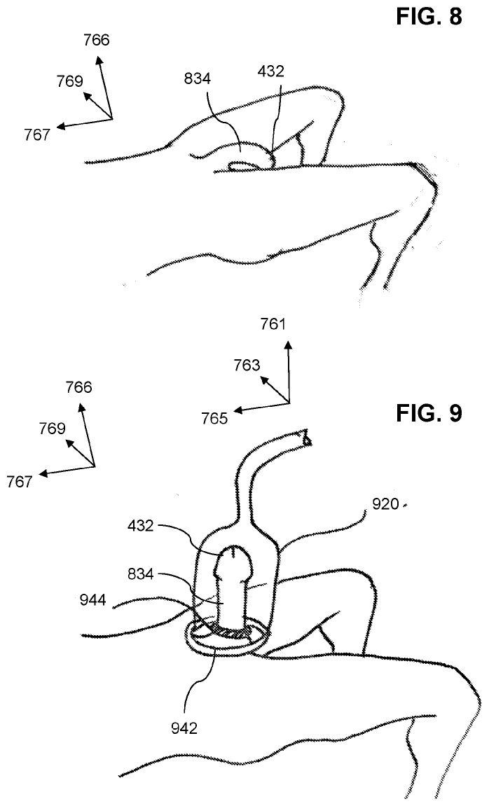

FIG. 9 is a schematic illustration of a device for treating impotence in accordance with an embodiment of the current invention. Optionally, a device may include comprising a tubular body 920. Optionally, the body is rigid and/or partially rigid. For example, the body 920 may have an opening 942 for insertion of penis into a lumen of the body 920. The opening may be configured for sealing and/or partially sealing over the pelvis and/or perineum of a user. Optionally the body 920 may be closed and/or connected to a vacuum at the other end. The tubular body 919 optionally includes one or more LIPUS transducers facing toward the lumen of the body 920. The tubular body may be connected to a vacuum. For example, in the figure, the device is shown fitted over the penis with its opening is snugly fit over the perineum and/or pelvis of a user. A partial vacuum is optionally created within the lumen of the body 920. The reduced pressure in the tubular body 920 optionally causes penile engorgement and/or tumescence and/or at least partial erection. At least part of the engorged penis (e.g. corpora cavernosa 834 and/or penis crura or roots) optionally come in intimate contact and/or vibrational communication with the LIPUS transducers. While in communication with tissue, the LIPUS transducers are activated in order to treat erectile dysfunction.

In some embodiments, a partial erection may be maintained during treatment by continuously applying vacuum or suction. Alternatively or additionally, erection may be maintained by applying a band 944 at the base of the penis after achieving full and/or partial erection. For example, erection may be achieved using drugs, devices, penile injection, and/or stimulation (e.g. self stimulation and/or applied vibration). Optionally, after completing the treatment, the suction and/or vacuum is released and/or the elastic band 944 is removed and/or the tubular body 920 is removed and the penis becomes flaccid again.

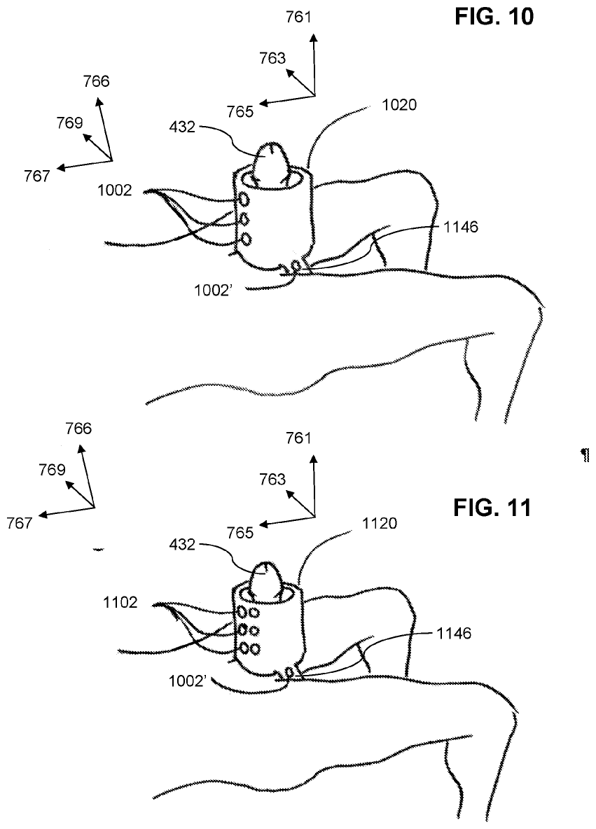

FIG. 10 is a schematic illustration of a device for treating impotence in accordance with an embodiment of the current invention. In some embodiments, a device for treating impotence includes a sheath 1020 including a lumen that can be fitted over the flaccid and/or partially erect penis. The sheath 1020 may fit over the body of the penis below the glans along all and/or part of the length of the penis. The device optionally includes one or more LIPUS transducers 1002 facing toward the lumen. In a particular embodiment, the transducers 1002 are situated in a row along the long axis of the sheath 1020. For example, the transducers 1002 may face the corpora cavernosa of the penis.

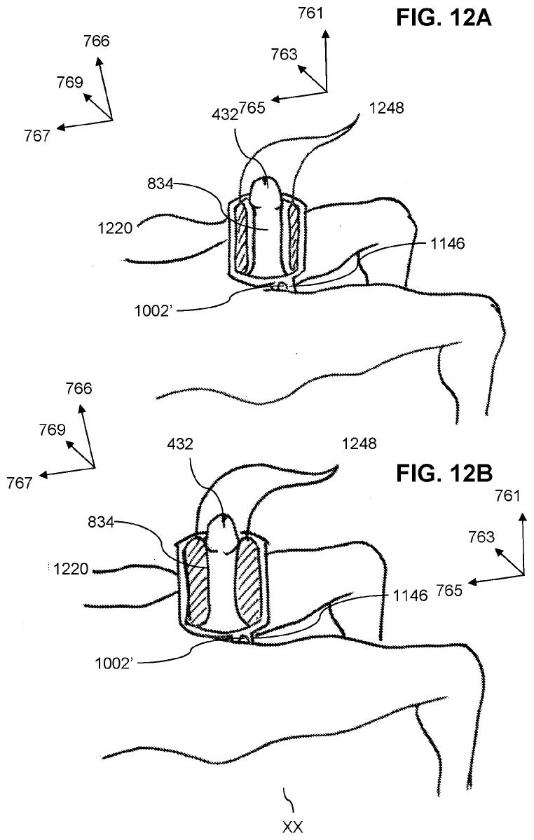

FIG. 11 is a schematic illustration of a device for treating impotence in accordance with an embodiment of the current invention. In a particular embodiment a sheath 1120 includes two or more rows of transducers 1102. For example, the transducers 1102 may face the lumen of the sheath 1120 and/or the corpora cavernosa.

In some embodiments (e.g. FIGS. 10-JJ, 4A-7C and/or 18-HH), the appliance comprises one, two or more extensions 1146 (e.g. 446, 1846). Optionally, the extensions 1146 are oriented along the axis of the device. Each extension 1146 is optionally provided with one or more transducers 1002′, 1102′. For example, these extensions 1146 may be extend proximally and/or away from the location of the transducers 1002, 1102 fitting over the corpora cavernosa. For example, the transducers 1002′ may fit snugly fit beside the scrotum and/or over the penis roots and/or to the ischiopubic rami. The transducers, situated on the extensions 1146 optionally face inward and/or posteriorly and/or cranially and/or are configured to make intimate contact with the penis roots (crura).

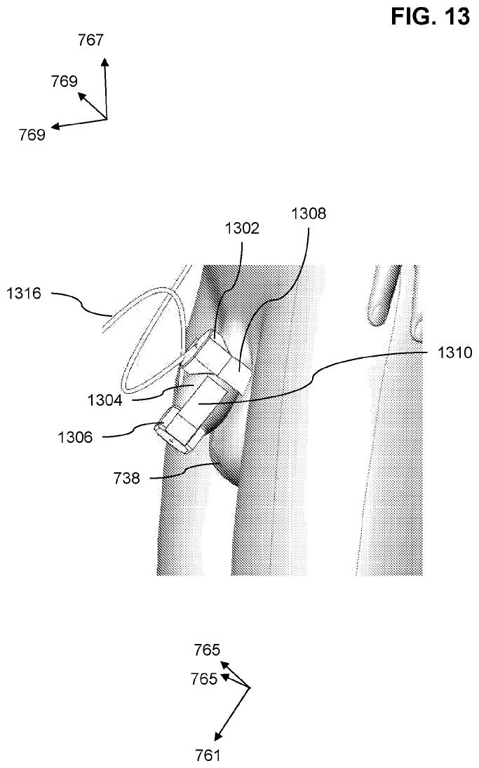

FIG. 12A is a schematic illustration of a system for treatment of impotence including an inflatable cushion in an uninflated state in accordance with an embodiment of the current invention. FIG. 12B is a schematic illustration of a system for treatment of impotence including an inflatable cushion in an inflated state in accordance with an embodiment of the current invention. In some embodiments, a sheath 1220 may be provided with a lumen including one or more inflatable cushions 1248. For example, the cushions 1248 may be inflated by filling with a fluid (e.g. air or water, for example as illustrated in FIG. 12B). These cushions 1248 may be inflated after fitting the sheath 1220 over the penis in order to bring the penis corpora cavernosa 834, and/or crura in intimate contact with transducers and/or to create a soft pleasant feeling to the penis skin. Intimate contact between a transducer and tissue may include actually pushing the transducer against the tissue and/or providing a transport medium between the tissue and/or the transducer for transmitting a signal from the transducer to the tissue.

In some embodiments, the cushions 1248 may be situated between the transducers and the penis cavernous corpora and/or crura and/or create an acoustic window. For example, the material for the acoustic windows may be selected to have a low attenuation or absorbance of the acoustic wave energy such as but not limited to: water, gels, silicone, elastomers, etc. Such material may serve also to dissipate the heat generated on the surface of the transducers by conduction and/or convection.

The appliance may made at least partially, of a heat conducting material such as but not limited to: metals, ceramics, heat conducting polymers or polymers with heat conducting metal particles or ceramics. These materials may permit to conduct and dissipate the heat generated at the surface of the transducers and prevent overheating penile tissue.

In some embodiments, the cushions 1248 may be configured for transmitting vibration (e.g. an ultrasound signal). For example, cushions 1248 may include a gel pad and/or a coupling pad. Optionally, cushions 1248 are made of and/or are covered by a distend-able or stretch-able elastomer such as but not limited to: polyurethane, silicone, etc. Optionally, after fitting the penis in the central lumen, the fluid filled cushions 1248 will be forced to bulge outwards at one or both ends and/or through slits or other openings in the body of the appliance. The pressure generated by this stretching when fitting the penis in the central lumen will hold the penis snugly, will prevent its slippage and/or will create intimate contact with the transducers and/or the acoustic windows.

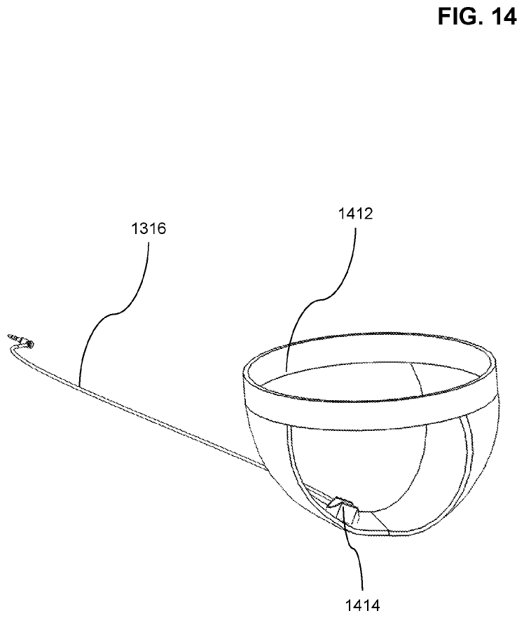

FIG. 13 is a schematic illustration of a system for treating impotence in use in accordance with an embodiment of the current invention. In some embodiments, an impotence treating system may include one or more transducers held in intimate contact with penile 1304 tissue. For example, the transducers may include, a low intensity ultrasound transducers for example a Low Intensity Pulsed Ultrasound LIPUS 1302 in intimate contact with the penile tissue 1304 and/or a vibrator 1306 in contact with the penile tissue 1304. For example, the LIPUS 1302 may be releasably attached to the ems and/or the corpora and/or the crura by a distensible cuff 1308. For example, vibrator 1306 may be held to the glans and/or reversible attached to the penile tissue 1304 by an elastic and/or clamping fastener and/or an adhesive fastener 1310. For example, an elastic and/or clamping fastener may include a bandage and/or a cuff and/or a clamp and/or a condom. For example, a distensible adhesive fastener 1310 may include adhering the transducer directly to the tissue 1304 with an adhesive chemical and/or a double sided adhesive pad and/or an adhesive tape for example to hold the transducer to the tissue. Optionally, a wire 1316 attaches the transducer to a power supply and/or a data storage device and/or a controller. The wire 1316 is optionally reversibly attached to the transducer 1414. Alternatively or additionally, the wire 1316 may be permanently attached to the transducer 1414. Alternatively or additionally, a transducer may include a wireless communication module and/or be in data connection to a data storage and/or a controller wirelessly. Alternatively or additionally, a transducer may include a wireless charging module and/or may be charged wirelessly.

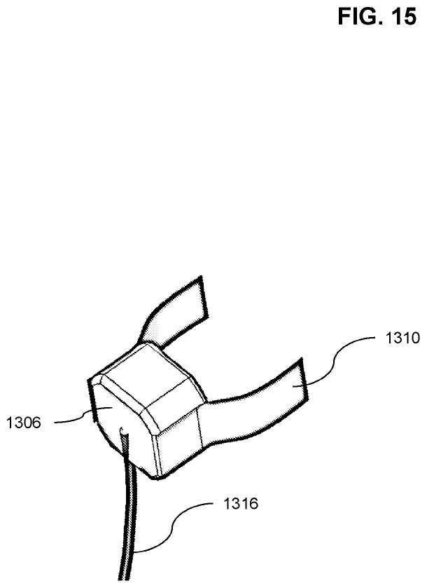

FIG. 14 is a schematic view of an appliance for attaching a transducer to penile tissue in accordance with an embodiment of the current invention. For example, a piece of underclothing 1412 for example, underwear a truss and/or belt may support a transducer 1414. Optionally, the transducer 1414 may be permanently attached to the underclothing 1412. Alternatively or additionally, the transducer 1414 may be reversibly attached to the underclothing 1412. Alternatively or additionally, a support structure in an undergarment may help position a transducer along with a body mounted holder. For example, a user may wear a body mounted device. For example, a support in underclothes for example as illustrated in FIG. 14 may push a transducer of a body mounted device (e.g. extensions 446 as illustrated in FIGS. 4A-7C) against the penile crura. Alternatively or additionally, a docking station for communication with and/or charging a device may be attached to clothes of a wearer.

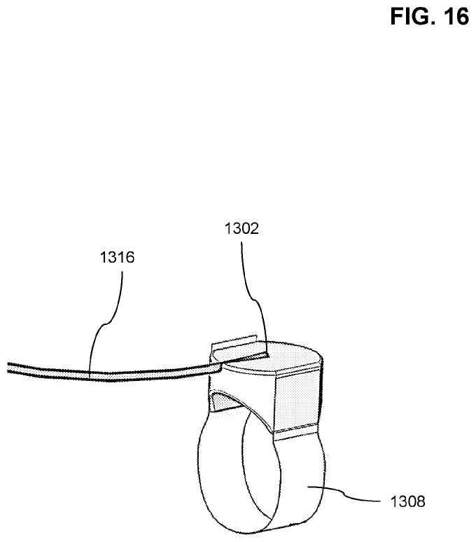

FIG. 15 illustrates a transducer 1306 and/or mount configured for reversable attachment to a glans 732 (e.g. as illustrated in FIGS. 7B, 7C) in accordance with an embodiment of the current invention. For example, the mount may use an adhesive patch 1310 and/or a condom and/or a cuff. In some embodiments, one, two or more vibratory modules are attached to the glans and/or just behind the glans 432 (e.g. as illustrated by vibrator 406 of FIG. 4). For example, the vibration may stimulate erectile response. Full or partial erection may be aroused for example, to improve contact with transducers and/or to improve penetration of ultrasound signals.

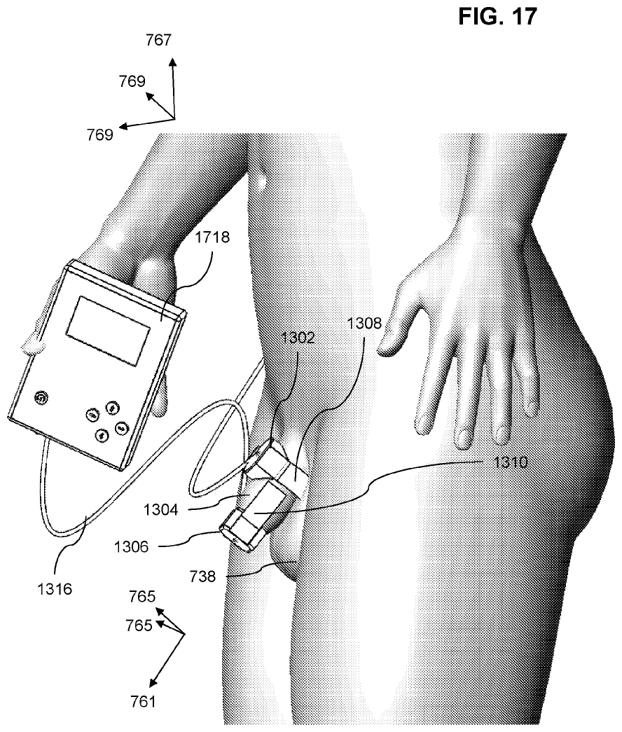

FIG. 16 illustrates a transducer 1302 and/or mount configured for reversible attachment to the penile corpora 834 in accordance with an embodiment of the current invention. Optionally, a mount uses a distend able cuff 1308. Alternatively or additionally, a transducer 1414 may be reversibly attached to the penile crura 736 using an appliance such as underclothes (e.g. underclothes 1412 as illustrated for example in FIG. 14) and/or a belt for bringing the transducer 1414 into intimate contact with crura 736.

In some embodiments a system may comprise sensor, for example a strain gauge. For example, sensor output may be used for assessing degree of tumescence and/or rigidity of erections. Sensor output may be used to asses the effects of treatment and/or adjust a treatment regime (e.g. an intensity, length and/or frequency of treatment) and/or treatment parameters e.g. a location of transducers and/or a frequency of an ultrasound signal and/or vibration etc.

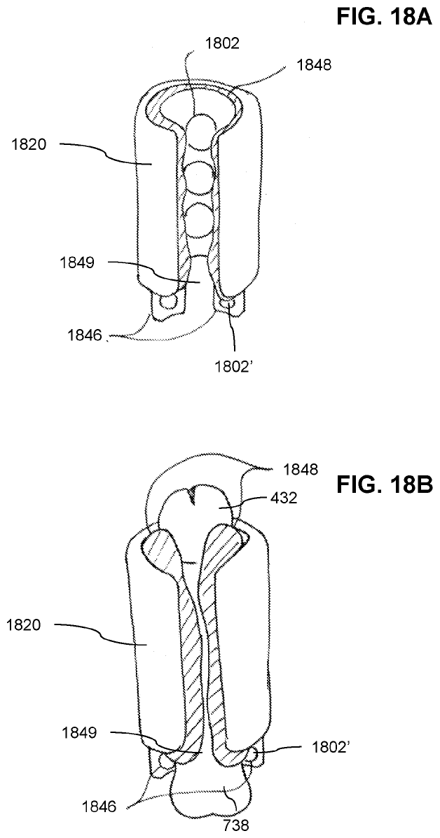

FIG. 17 illustrates a control system for an erectile disfunction treatment system in accordance with an embodiment of the current invention. For example, any, some or all of a low intensity ultrasound module 1302, vibratory module 1306 and/or strain gauge may be connected to a portable control module 1718. For example, a strain gauge may be built into and/or attached to a cuff 1308, for example to measure tumescence. Alternatively or additionally, a gauge may be built into a longitudinally oriented member (e.g. between adhesive pads on an adhesive strip 1310), for example, to measure elongation and/or rigidity. For example, sensor output may serve as feedback mechanism to adjust parameters of ultrasound application and/or application of vibration.

In some embodiments, an intensity ultrasound transducer may deliver between 40 mW to 500 mW/cm2. In case of attaining a predetermined degree of tumescence and/or rigidity the vibratory module may be turned off and/or modulated. Optionally, the low intensity ultrasound modules may act to improve potency in long term. In some embodiments, the treatment may cure impotency. Optionally, the Ultrasound signal has a predominantly non-thermal effect. For example, the effect may include angiogenesis, vascularization, changes in elasticity and/or Nitrogen Oxide synthesis NOS and/or other metabolic pathways.

In some embodiments, a low intensity ultrasound transducer (e.g. transducer 1302, 402a and/or 402b) may apply a signal at a frequency between 0.1 to 1 MHz and/or between 1 and 3 MHz and/or between 3 to 10 MHz. Optionally, the on/off duty cycle ratio may range between 0.5 to 1 and/or between 1 to 1:8 and/or between 1.8 to 4. Optionally, the frequency of the duty cycles may be between 1 to 50 Hz and/or between 50 Hz to 500 Hz and/or between 500 Hz to 5 kHz and/or between 5 kHZ to 50 kHz.

In some embodiments, the frequency of vibration of a vibratory transducer (e.g. transducer 1306 and/or 406) may vibrate with a frequency ranging between 15 to 300 Hz.

In a particular embodiment, contact between a transducer and penile tissue may be enhanced for example using a fluid filled cushion and/or a gel and/or fluid that may be delivered between the transducer and penile tissue. Alternatively or additionally, a Ultra Sound conveying pad may be positioned between a transducer and the penile tissue.

In some embodiments, the control module 1718 may include a hand held electronic device such as: cellular phone, smart watch, laptop, iPad, PDA. The connection may be wired and/or wireless. Optionally, the control module 1718 may include a program module. For example, the control module 1718 may be configured for receiving sensor output, analyzing data, sending control signals to transducers, receiving data from transducers and/or receive user input (for example over a user interface) and/or displaying data (for example over a user interface), and/or presenting warning and/or presenting instructions to the user.

In some embodiments, data from different users may be made anonymous and/or evaluated by cloud computing. For example, the evaluation may be used in order to improve the treatment parameters and to individualize treatment.

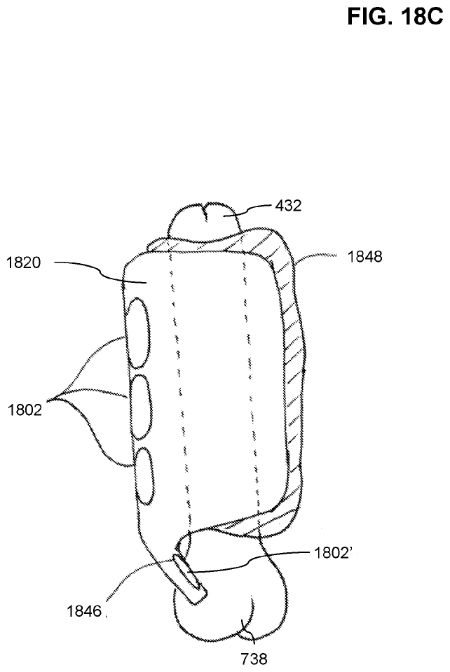

FIG. 18A is a schematic illustration of a dorsal side of a device for treatment of impotence including a sheath having a dorsal slit and/or an inflatable cushion in an empty state in accordance with an embodiment of the current invention. FIG. 18B is a schematic illustration of a dorsal side of a device for treatment of impotence including a sheath having a dorsal slit and/or an inflatable cushion in an inflated state in accordance with an embodiment of the current invention. FIG. 18C is a schematic lateral view illustration of a device for treatment of impotence including a sheath having a dorsal slit and/or an inflatable cushion in an inflated state in accordance with an embodiment of the current invention. In some embodiments, the appliance is provided with a longitudinal groove 1849 communicating with the central lumen. Optionally, the groove 1849 facilitates introducing the penis into the lumen of the appliance. Optionally, once the penis is inserted into the slit 1849, a cushion 1848 brings it into intimate contact with transducers 1802. The penis may be in the flaccid fully erect and/or partially erect state. The appliance may fit over part or along the entire length of the body of the penis. Optionally, the glans of the penis protrudes beyond distal opening of the central lumen. Optionally, the protruding glans may prevent slippage of the penis from the appliance.

In some embodiment, there are one or more extensions 1846 extending proximally from the sheath 1820. For example, the extension 1846 may be extend from a ventral side of the sheath 1820. Optionally, the extensions 1846 have cranial directed transducers. For example, the extensions may be configured to bring the transducers 1802′ into intimate contact with a crura and/or root of the penis.

In a particular embodiment, the inner lining of the central lumen may be sticky and prevent the penis from slipping away. Such lining may be of a sticky soft elastomer such as but not limited to: silicone, polyurethane or a contact adhesive gel.

In a particular embodiment, a system for treating impotence is described.

It is expected that during the life of a patent maturing from this application many relevant technologies (e.g. sensors, transducers) will be developed and the scope of the terms is intended to include all such new technologies a priori.

As used herein the term “about” refers to ±10% The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. When multiple ranges are listed for a single variable, a combination of the ranges is also included (for example the ranges from 1 to 2 and/or from 2 to 4 also includes the combined range from 1 to 4).

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for treating impotence comprising:
a sheath having a lumen configured to fit around a penis;
a transducer directed towards said lumen;
at least one more transducer directed towards a penile crura located proximal to said lumen.

2. The system of claim 1, wherein said at least one more transducer directed to the penile crura is directed at least partially in a dorsal direction with respect to said sheath.

3. The system of claim 1, wherein said at least one more transducer includes a pair of inward directed transducers.

4. A system for treating impotence comprising:
a sheath having a lumen configured to fit around a penis;
a transducer directed towards said lumen;
a proximal extension from said sheath;
at least one more transducer held by said extension.

5. The system of claim 4, wherein the sheath is elastic for accommodating different penile diameters.

6. The system of claim 4, wherein said at extension is configured to hold said at least one more transducer against a crura of said penis when at least a portion of a body said penis is inside said lumen.

7. The system of claim 4, further comprising a vibrator.

8-27. (canceled)

28. The system of claim 4, wherein said sheath further includes a longitudinal slit.

29-31. (canceled)

32. The system of claim 4, wherein the lumen is provided with an adhesive surface to prevent slippage of the penis.

33. A method for treating erectile disfunction comprising:
applying over a penis of an appliance comprising at least one LIPUS transducer positioned in intimate contact with penile tissue of a body of the penis;
applying at least one more LIPUS transducer positioned in intimate contact with a crura of the penis; and
activating the LIPUS transducer and the at least one more LIPUS transducer for treating or improving erectile dysfunction.

34. The method of claim 33, wherein said at least one LIPUS transducer and said at least one more LIPUS transducer are applied simultaneously.

35. The method of claim 33, further comprising:
creating penis engorgement resulting in at least a partial erection.

36. The method of claim 33, further comprising:
retaining said transducer in contact with said the penile tissue with a sheath enveloping at least part of said body of said penis.

37. The method of claim 36, further comprising:
retaining said at least one more transducer in contact with said crura with a proximal extension from said sheath.

38. The method of claim 33, further comprising:
discern a quality of contact between a transducer and tissue.

39. The method of claim 38, further comprising:
prevent said activating when said quality of contact is poor.

40. The method of claim 33, wherein activating includes delivering between 20 to 500 W/cm².

41. The method of claim 33, wherein activating includes delivering a signal of frequency between 1 to 3 MHz.

42. The method of claim 33, wherein activating includes delivering signals at a burst cycle frequency of 500 Hz to 2 KHz.

43. The method of claim 33, wherein activating includes delivering a vibratory signal of frequency between 10 to 300 Hz.

44. (canceled)
